(19) United States
(12) Patent Application Publication
(10) Pub. No.: US 2005/0244898 A1
(43) Pub. Date: Nov. 3, 2005

(54) Large-area two-dimensional non-adhesive cell arrays for sensing and cell-sorting applications

(76) Inventors: Robert E. Cohen, Jamaica Plain, MA (US); Paula T. Hammond-Cunningham, Newton, MA (US); Darrell J. Irvine, Arlington, MA (US); Heejae Kim, Cambridge, MA (US); Junsang Doh, Cambridge, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/988,976

(22) Filed: Nov. 15, 2004

Related U.S. Application Data
(60) Provisional application No. 60/523,399, filed on Nov. 19, 2003.
(60) Provisional application No. 60/532,686, filed on Dec. 24, 2003.

(51) Int. Cl. G01N 33/53; G01N 33/567; C12M 1/34

(52) U.S. Cl. 435/7.2; 435/287.2

(57) ABSTRACT

One aspect of the present invention relates to an array of non-adhesive cells, comprising a polymeric substrate, an array of a graft copolymer bound to the polymeric substrate, an antibody bound to the polymeric substrate in an area of the polymeric substrate not covered by the graft copolymer, and a non-adhesive cell bound to the antibody. Another aspect of the present invention relates to a method of preparing an array of non-adhesive cells, comprising depositing an array of a graft copolymer upon a polymeric substrate; binding an antibody to an area of the polymeric substrate not covered by the graft copolymer; and binding a non-adhesive cell to the antibody.

### RELATED APPLICATIONS

This application claims the benefit of priority to U.S. Provisional Patent Application Ser. No. 60/523,399, filed Nov. 19, 2003; and U.S. Provisional Patent Application Ser. No. 60/532,686, filed Dec. 24, 2003; the specifications of which are hereby incorporated in their entirety.

### BACKGROUND OF THE INVENTION

There is currently great interest in the design of imaging-based high-throughput cellular analysis systems, platforms for rare-event detection, ultrasensitive cell-based biosensors, and lab-on-a-chip devices. Taylor, D. L.; Woo, E. S.; Giuliano, K. A. Curr. Opin. Biotechnol. 2001, 12, 75-81; Kapur, R.; Giuliano, K. A.; Campana, M.; Adams, T.; Olson, K.; Jung, D.; Mrksich, M.; Vasudevan, C.; Taylor, D. L. Biomed. Microdevices 1999, 2, 99-109; Kraeft, S. K.; Sutherland, R.; Gravelin, L.; Hu, G. H.; Ferland, L. H.; Richardson, P.; Elias, A.; Chen, L. B. Clin. Cancer Res. 2000, 6, 434-442; Rider, T. H.; Petrovick, M. S.; Nargi, F. E.; Harper, J. D.; Schwoebel, E. D.; Mathews, R. H.; Blanchard, D. J.; Bortolin, L. T.; Young, A. M.; Chen, J. Z.; Hollis, M. A. Science 2003, 301, 213-215. These technologies, as well as fundamental studies of cell biology, could be greatly facilitated by the use of screening surfaces that selectively immobilize cells with arbitrary characteristics into defined arrays on a 2D surface. Whitesides, G. M.; Ostuni, E.; Takayama, S.; Jiang, X. Y.; Ingber, D. E. Annu. Rev. Biomed. Eng. 2001, 3, 335-373. While techniques for patterning adherent cells have been extensively investigated, such methods have not been accessible to non-adherent cells, such as lymphocytes or stem/progenitor cells.

The idea of patterning cells onto surfaces has become more prevalent as various patterning methods have emerged over the past several years. Folch, A.; Toner, M. Annu. Rev. Biomed. Eng. 2000, 2, 227-256. Most of these studies have focused on fibroblast or endothelial cells, with some exceptions such as nerve cells and liver cells. Michel, R.; Lussi, J. W.; Csucs, G.; Reviakine, I.; Danuser, G.; Ketterer, B.; Hubbell, J. A.; Textor, M.; Spencer, N. D. Langmuir 2002, 18, 3281-3287; Welle, A.; Gottwald, E. Biomed. Microdevices 2002, 4, 33-41; Csucs, G.; Michel, R.; Lussi, J. W.; Textor, M.; Danuser, G. Biomaterials 2003, 24, 1713-1720; Hyun, J. H.; Ma, H. W.; Zhang, Z. P.; Beebe, T. P.; Chilkoti, A. Adv. Mater. 2003, 15, 576-579; Chen, C. S.; Mrksich, M.; Huang, S.; Whitesides, G. M.; Ingber, D. E. Science 1997, 276, 1425-1428; Andersson, A. S.; Backhed, F.; von Euler, A.; Richter-Dahlfors, A.; Sutherland, D.; Kasemo, B. Biomaterials 2003, 24, 3 427-3436; T an, W.; Desai, T. A. Tissue Eng. 2003, 9, 255-267; Craighead, H. G.; James, C. D.; Turner, A. M. P. Curr. Opin. Solid State Mat. Sci. 2001, 5, 177-184; Chang, J. C.; Brewer, G. J.; Wheeler, B. C. Biomaterials 2003, 24, 2863-2870; Welle, A.; Gottwald, E. Biomed. Microdevices 2002, 4, 33-41; Yamato, M.; Konno, C.; Utsumi, M.; Kikuchi, A.; Okano, T. Biomaterials 2002, 23, 561-567. The patterning of these tissue-forming cells has been performed via techniques utilizing adhesion receptor ligands, such as fibronectin or RGD peptides, or non-specific adhesive interactions with a number of organic surfaces. Michel, R.; Lussi, J. W.; Csucs, G.; Reviakine, I.; Danuser, G.; Ketterer, B.; Hubbell, J. A.; Textor, M.; Spencer, N. D. Langmuir 2002, 18, 3281-3287; Csucs, G.; Michel, R.; Lussi, J. W.; Textor, M.; Danuser, G. Biomaterials 2003, 24, 1713-1720; Hyun, J. H.; Ma, H. W.; Zhang, Z. P.; Beebe, T. P.; Chilkoti, A. Adv. Mater. 2003, 15, 576-579; Chen, C. S.; Mrksich, M.; Huang, S.; Whitesides, G. M.; Ingber, D. E. Science 1997, 276, 1425-1428; Tan, W.; Desai, T. A. Tissue Eng. 2003, 9, 255-267; Craighead, H. G.; James, C. D.; Turner, A. M. P. Curr. Opin. Solid State Mat. Sci. 2001, 5, 177-184; Chang, J. C.; Brewer, G. J.; Wheeler, B. C. Biomaterials 2003, 24, 2863-2870. On the other hand, important cell types such as stem cells, lymphocytes, and certain tumor cells are weakly adherent or non-adherent. Handgretinger, R.; Gordon, P. R.; Leimig, T.; Chen, X.; Buhring, H.-J.; Niethammer, D.; Kuci, S. Annals of the New York Academy of Science 2003, 996, 141-151; Eggermann, J.; Kliche, S.; Jarmy, G.; Hoffinann, K.; Mayr-Beyrle, U.; Debatin, K. M.; Waltenberger, J.; Beltinger, C. Cardiovasc. Res. 2003, 58, 478-486; Wilson, H. L.; O'Neill, H. C. Immunol. Cell Biol. 2003, 81, 144-151; Sroka, J.; von Gunten, M.; Dunn, G. A.; Keller, H. U. Int. J. Biochem. Cell Biol. 2002, 34, 882-899. The isolation of non-adherent cells in a 2D surface array has not yet been reported, and presents new challenges. Arrays of lymphocytes could be particularly useful for controlling the cell-cell contacts that dictate immune cell function while excluding influences from interactions between the neighboring cells of the same type. Arrays of lymphocytes also present new possibilities for ultrasensitive and rapid-detection biosensors. Rider, T. H.; Petrovick, M. S.; Nargi, F. E.; Harper, J. D.; Schwoebel, E. D.; Mathews, R. H.; Blanchard, D. J.; Bortolin, L. T.; Young, A. M.; Chen, J. Z.; Hollis, M. A. Science 2003, 301, 213-215.

### SUMMARY OF THE INVENTION

In one embodiment, the present invention relates to an array of non-adhesive cells comprising:
a) a polymeric substrate;
b) an array of a graft copolymer bound to the polymeric substrate;
c) an antibody bound to the polymeric substrate in an area of the polymeric substrate not covered by the graft copolymer; and
d) a non-adhesive cell bound to the antibody.

In a further embodiment, the polymeric substrate comprises bilayers of two different polymers. In a further embodiment, the polymeric substrate comprises bilayers of two different polymers, wherein one polymer is linear poly(ethylenimine) and the other one is poly(acrylic acid) (PAA). In a further embodiment, the graft copolymer comprises poly(allylamine). In a further embodiment, the graft copolymer comprises poly(ethylene glycol). In a further embodiment, the graft copolymer is poly(allylamine)-g-poly(ethylene glycol). In a further embodiment, the graft copolymer is deposited on the polymeric substrate by polymer-on-polymer stamping (POPS). In a further embodiment, the antibodies are CD44:FITC. In a further embodiment, the non-adhesive cells are lymphocyte or stem cells. In a further embodiment, the non-adhesive cells are B cells. In a further embodiment, the non-adhesive cells are CH27 B cells.

In another embodiment, the present invention relates to an array of non-adhesive cells, comprising:
a) a polymeric substrate;
b) an array of a biotinylated graft copolymer bound to the polymeric substrate, wherein the biotin is bound to a protein having a high affinity for biotin;
c) a graft copolymer free of biotin bound to an area of the polymeric substrate not covered by the array of biotinylated graft copolymer;
d) a biotinylated antibody bound to the protein; and
e) a non-adhesive cell bound to the antibody.

In a further embodiment, the polymeric substrate comprises bilayers of two different polymers. In a further embodiment, the polymeric substrate comprises bilayers of two different polymers, wherein one polymer is linear poly(ethylenimine) and the other one is poly(acrylic acid) (PAA). In a further embodiment, the biotinylated graft copolymer comprises poly(allylamine). In a further embodiment, the biotinylated graft copolymer comprises poly(ethylene glycol). In a further embodiment, the biotinylated graft copolymer is biotinylated poly(allylamine)-g-poly(ethylene glycol). In a further embodiment, the biotinylated graft copolymer is deposited on the polymeric substrate by polymer-on-polymer stamping (POPS). In a further embodiment, the graft copolymer free of biotin comprises poly(allylamine). In a further embodiment, the graft copolymer free of biotin comprises poly(ethylene glycol). In a further embodiment, the graft copolymer free of biotin is poly(allylamine)-g-poly(ethylene glycol). In a further embodiment, the protein having a high affinity for biotin is streptavidin. In a further embodiment, the antibody is CD44:FITC. In a further embodiment, the non-adhesive cells are lymphocyte or stem cells. In a further embodiment, the non-adhesive cells are B cells. In a further embodiment, the non-adhesive cells are CH27 B cells.

In another embodiment, t he p resent i nvention r elates t o a m ethod o f p reparing a non-adhesive cell array, comprising:
a) depositing an array of a graft copolymer upon a polymeric substrate;
b) binding an antibody to an area of the polymeric substrate from step a) not covered by the graft copolymer; and
c) binding a non-adhesive cell to the antibody from step b).

In a further embodiment, depositing the array of graft copolymer comprises POPS.

In a further embodiment, binding an antibody to the polymeric substrate comprises immersing the polymeric substrate from step a) into a solution of an antibody followed by rinsing.

In a further embodiment, binding a non-adhesive cell to the antibody comprises placing a suspension of the non-adhesive cell over the polymeric substrate from step b); allowing the non-adhesive cell to precipitate upon the polymeric substrate; and inverting the polymeric substrate allowing the non-bound non-adhesive cells to fall off.

In a further embodiment, depositing the array of graft copolymer comprises POPS; binding anitibodies to the polymeric substrate comprises immersing the polymeric substrate from step a) into a solution of antibodies followed by rinsing; and binding non-adhesive cells to the antibodies comprises placing a suspension of the non-adhesive cells over the polymeric substrate from step b); allowing the non-adhesive cells to precipitate down upon the polymeric substrate; and inverting the polymeric substrate allowing the non-bound non-adhesive cells to fall off.

In a further embodiment, the present invention relates to the method of preparing an array of non-adhesive cells as described in steps a)-c) above, wherein the polymeric substrate comprises bilayers of two different polymers. In a further embodiment, the polymeric substrate comprises bilayers of two different polymers, wherein one polymer is linear poly(ethylenimine) and the other one is poly(acrylic acid) (PAA). In a further embodiment, the graft copolymer comprises poly(allylamine). In a further embodiment, the graft copolymer comprises poly(ethylene glycol). In a further embodiment, the graft copolymer is poly(allylamine)-g-poly(ethylene glycol). In a further embodiment, the antibody is CD44:FITC. In a further embodiment, the non-adhesive cells are lymphocyte or stem cells. In a further embodiment, the non-adhesive cells are B cells. In a further embodiment, the non-adhesive cells are CH27 B cells.

In another embodiment, the present invention relates to a method of preparing an non-adhesive cell array, comprising:
a) depositing an array of a graft copolymer upon a polymeric substrate;
b) biotinylating the graft copolymer from step a);
c) depositing a graft copolymer upon an area of the polymeric substrate from step b) not covered by the biotinylated graft copolymer;
d) binding a protein that has a high affinity for biotin to the biotinylated graft copolymer from step c);
e) binding a biotinylated antibody to the protein from step d); and
f) binding a non-adhesive cell to the antibody from step e).

In a further embodiment, depositing the array of graft copolymer comprises POPS.

In a further embodiment, biotinylating the graft copolymer comprises placing a solution of sulfo-NHS-LC-biotin over the graft copolymer from step a) followed by rinsing.

In a further embodiment, depositing the graft copolymer upon the area of the polymeric substrate from step b) not covered by the biotinylated graft copolymer comprises immersing the polymer substrate into a solution of the graft copolymer followed by rinsing and blow drying.

In a further embodiment, binding a protein that has a high affinity for biotin to the biotinylated graft copolymer from step c) comprises immersing the polymer substrate from step c) into a solution of the protein followed by rinsing.

In a further embodiment, binding biotinylated antibodies to the protein from step d) comprises immersing the polymeric substrate from step d) into a solution of biotinylated antibodies followed by rinsing.

In a further embodiment, binding non-adhesive cells to the antibodies from step e) comprises placing a suspension of the non-adhesive cells over the polymeric substrate from step e); allowing the non-adhesive cells to precipitate down upon the polymeric substrate; and inverting the polymeric substrate allowing the non-bound, non-adhesive cells to fall off.

In a further embodiment, the non-adhesive cells are biotinylated.

In a further embodiment, the present invention relates to the method of preparing an array of non-adhesive cells, wherein:
i. depositing the array of graft copolymer comprises POPS;
ii. biotinylating the graft copolymer comprises placing a solution of sulfo-NHS-LC-biotin over the graft copolymer from step a) followed by rinsing;
iii. depositing the graft copolymer upon areas of the polymeric substrate from step b) not covered by the biotinylated graft copolymer comprises immersing the polymer substrate into a solution of the graft copolymer followed by rinsing and blow drying;
iv. binding a protein that has a high affinity for biotin to the biotinylated graft copolymer from step c) comprises immersing the polymer substrate from step c) into a solution of the protein followed by rinsing;
v. binding biotinylated antibodies to the protein from step d) comprises immersing the polymeric substrate from step d) into a solution of biotinylated antibodies followed by rinsing; and
vi. binding non-adhesive cells to the antibodies from step e) comprises placing a suspension of the non-adhesive cells over the polymeric substrate from step e); allowing the non-adhesive cells to precipitate down upon the polymeric substrate; and inverting the polymeric substrate allowing the non-bound, non-adhesive cells to fall off. In a further embodiment, the non-adhesive cells are biotinylated.

In a further embodiment, the present invention relates to the method of preparing an array of non-adhesive cells as described in steps a).-f) above, wherein the polymeric substrate comprises bilayers of two different polymers. In a further embodiment, the polymeric substrate comprises bilayers of two different polymers, wherein one polymer is linear poly(ethylenimine) and the other one is poly(acrylic acid) (PAA). In a further embodiment, the graft copolymer comprises poly(allylamine). In a further embodiment, the graft copolymer comprises poly(ethylene glycol). In a further embodiment, the graft copolymer is poly(allylamine)-g-poly(ethylene glycol). In a further embodiment, the antibody is CD44:FITC. In a further embodiment, the non-adhesive cells are lymphocyte or stem cells. In a further embodiment, the non-adhesive cells are B cells. In a further embodiment, the non-adhesive cells are CH27 B cells. In a further embodiment, the non-adhesive cells are biotinylated.

In another embodiment, the present invention relates to a biosensor comprising an array of non-adhesive cells.

These embodiments of the present invention, other embodiments, and their features and characteristics, will be apparent from the detailed description, claims and figures that follow.

### BRIEF DESCRIPTION OF THE FIGURES

FIG. 1 depicts schematically the polymer-on-polymer stamping (POPS) process used in certain embodiments of the present invention.

FIG. 2 depicts the results of protein adsorption experiments as determined by surface plasmon resonance.

FIG. 3 depicts fabrication of antibody and B cell array by simple adsorption of antibody. (a) Schematic procedure of antibody array template fabrication. (b) Patterned array of fluorescence labeled antibody. (c) B cell array fabricated with antibody array template shown in (b).

FIG. 4 depicts fabrication of biotinylated antibody and B cell array. (a) Schematic procedure of fabrication of patterned array of biotinylated antibody. (b) Array of fluorescence labeled antibody fabricated by biotin-streptavidin conjugation. (c) B cell array fabricated from patterned array of biotinylated antibody shown in (b).

FIG. 5 depicts schematically a method of fabrication of a B cell array on an antibody array template.

FIG. 6 depicts schematically the fabrication of a cellular array of biotinylated B cells.

FIG. 7 depicts B cell arrays for several different antibody array templates.

### DETAILED DESCRIPTION OF THE INVENTION

Overview

Surprisingly, an approach to generate patterns of non-adherent cells, e.g., B lymphocytes, in single-cell arrays over cm² areas of polymer-coated substrates for pathogen detection and immunological applications has been discovered. The approach is applicable to a broad range of culture surfaces, provides high-fidelity cellular patterns over entire culture surfaces with simple seeding and washing, and can be extended and generalized to many cell types.

Remarkably, a reliable method to produce isolated B cell arrays utilizing nonlithographic microscopic patterning over large areas and a novel functionalizable, protein adsorption-resistant copolymer has been discovered. Due to the inherent weak cell-substrate adhesion displayed by B cells, specific antibodies or streptavidin-biotin were used to facilitate the immobilization of B cells into an array. Surfaces were chosen for which the attachment of cell-binding molecules to the surface via a spacer group allows free orientation of the antibody. The cellular array used, e.g., a patterned array of antibodies with optimized binding strength to a single cell, alternating with a non-adhesive, cytophobic surface.

The discovery of a simple means of patterning antibodies for specific dendritic or antigen-presenting cell systems will enable the creation of arrays for immobilization of a large number of cells relevant to, e.g., the immune system. Moreover, this capability establishes the viability of the use of polymer stamping as a means of biofimctional patterning for a wide range of applications.

Introduction

As various patterning techniques have emerged, the idea of patterning biological systems, such as nucleic acids, proteins or cells, has become more and more prevalent for numerous applications, such as biochips or biosensors. Surface patterning for biological applications ranges from topographical patterning to chemical patterning, and the applied techniques vary depending on the goals of the studies. Microcontact printing has been widely used to create patterns of alternating chemical surface functionality; self-assembled monolayers (SAMs) of various functionalities have been used to comprise surfaces with patterned biofunctionality, either through selective adsorption of a protein, or direct covalent immobilizations of biomolecules on the microcontact printed surface or direct stamping of proteins. Such arrays have been used to template cellular arrays aimed at understanding critical issues in cell biology, such as motility and apoptosis.

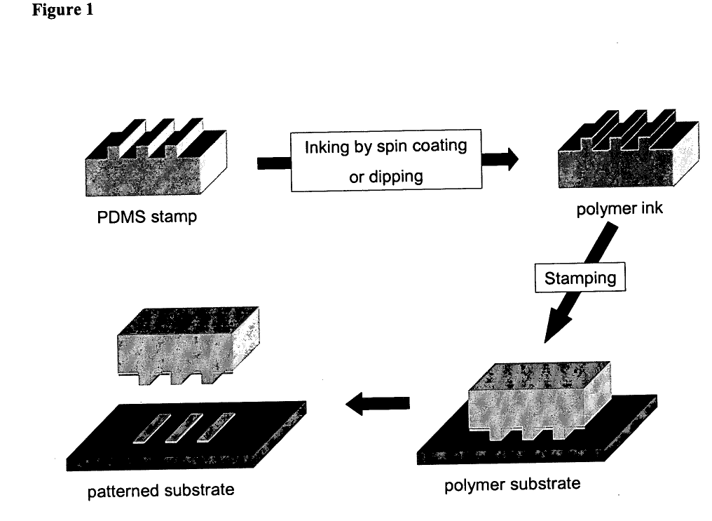

Compared to SAM-based microcontact printing techniques that utilize thiol, siloxane or other small molecules, the polymer-on-polymer stamping (POPS) method has advantages in the flexibility of both substrate and ink selection because molecular transfer can occur in association with electrostatic, van der Waals, or hydrogen-bonding interactions as well as covalent bonding; furthermore, the multivalent nature of polymer systems allows the use of weaker interactions while still maintaining a stable monolayer. For these reasons, POPS as depicted in FIG. 1, is a universal approach, particularly when combined with the use of polyelectrolyte multilayers as base layers on various kinds of planar and nonplanar substrates. The ability to tailor the functionality and surface composition of polyelectrolyte multilayers opens a wider range of potential polymer “inks”. In addition, the molecular conformation of the stamped layer in the POPS of weak polyelectrolytes can be tuned with the adjustment of ink pH, just like the molecular conformation within the multilayer film, which provides a very simple way to control the extent of functionalization in surface reactions, as reported previously. In the study, it was shown that the thickness of the poly(allylamine hydrochloride) (PAH) layer transferred by POPS changed with the pH of PAH ink due to the change of molecular conformation and, consequently, the number of RGD oligopeptides reacted with PAH layer varied with the number of free amine groups at the given molecular conformation.

On the other hand, the need for materials that are resistant to the non-specific adsorption of proteins or other biomolecules, and yet relatively stable, has drawn great attention from many researchers in bio-related areas. Furthermore, it has become an important issue to render the surface bioactive via immobilization of biomolecules without non-specific adsorption of unwanted substances. Of many materials reported to be resistant to non-specific protein adsorption, one of the most studied and most exploited is poly(ethylene glycol) (PEG). Various methods have been reported to create a stable coating of PEG on substrates, including simple adsorption, surface grafting, chemical cross-linking, plasma polymerization, and self-assembled monolayer formation. In addition, the functionalization of PEG homo or copolymers with biomolecules, such as oligopeptides, glucoses, and proteins, can be performed to generate bio-specific surfaces. While PEG domain generates bio-inert surface with resistance to non-specific protein adsorption, the copolymer enhances binding with surface and thus stability of the coated surface.

Herein a new graft copolymer, poly(allylamine)-g-poly(ethylene glycol) is synthesized to satisfy multiple demands. A weak polycationic backbone of poly(allylamine) has long been studied in association with polymer multilayers and POPS. It is demonstrated that the attachment of PEG side chains to this hydrophobic weak polycation yields additional features of protein adsorption resistance, in addition to all the advantages of weak polyelectrolytes, such as a tunable monolayer thickness. The graft copolymer was used as an ink for POPS to generate micron-scale, long-range patterns with high fidelity, and subsequent biotin or maleimide functionalization was carried out through an amine-based surface reaction. Employment of the surface reaction enabled the same surface to be functionalized with many different materials without making all derivatives by tedious synthesis and purification steps. The functionalized surface was used as a template for the fabrication of a B cell array. The versatility of the graft copolymer provided the flexibility to optimize the template characteristics and a clean array of single B cells was obtained over large areas using this approach.

### DEFINITIONS

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The articles “a” and “an” are used herein to refer to one or to more than one (i.e., to at least one) of the grammatical object of the article. By way of example, “an element” means one element or more than one element.

The terms “comprise” and “comprising” are used in the inclusive, open sense, meaning that additional elements may be included.

The term “including” is used to mean “including but not limited to”. “Including” and “including but not limited to” are used interchangeably.

The term “lymphocyte” is used to mean any of the nearly colorless cells found in the blood, lymph, and lymphoid tissues, constituting approximately 25 percent of white blood cells and including B cells, which function in humoral immunity, and T cells, which function in cellular immunity.

The term “B cells” is used to mean one of the two major classes of lymphocytes produced in bone marrow that are involved in antibody production.

The term “antibody” is used to mean molecules that are plasma proteins that bind specifically to particular molecules known as antigens. Antibody molecules are produced in response to immunization with antigen. They are specific molecules of the humoral immune response that bind to and neutralize pathogens or prepare them for uptake and destruction by phagocytes.

The term “array” is used to mean an intended pattern.

The term “non-adhesive cells” is used to mean those cells, such as lymphocytes or stem cells, that have diminished adhesive properties to a substrate as compared to those cells known to be adhesive cells.

The term “polymer” is used to mean a large molecule formed by the union of repeating units (monomers). The term polymer also encompasses copolymers.

The term “copolymer” is used to mean a polymer of two or more different monomers.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “heteroatom” is art-recognized and refers to an atom of any element other than carbon or hydrogen. Illustrative heteroatoms include boron, nitrogen, oxygen, phosphorus, sulfur and selenium.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl”, “heteroaryl”, or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂⁻. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 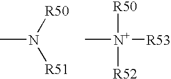
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 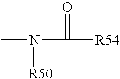
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 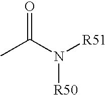
wherein R50 and R51 are as defined above. Certain embodiments of the amide in the present invention will not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carboxyl” is art recognized and includes such moieties as may be represented by the general formulas: 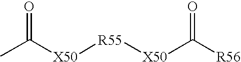
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R6 1, where m and R61 are defined above. Where X50 is an oxygen and R55 or R56 is not hydrogen, the formula represents an “ester”. Where X50 is anoxygen, and R55 is as defined above, the moietyis referredto herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The term “carbamoyl” refers to —O(C═O)NRR′, where R and R′ are independently H, aliphatic groups, aryl groups or heteroaryl groups.

The term “oxo” refers to a carbonyl oxygen (═O).

The terms “oxime” and “oxime ether” are art-recognized and refer to moieties that may be represented by the general formula: 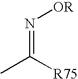
wherein R75 is hydrogen, alkyl, cycloalkyl, alkenyl, alkynyl, aryl, aralkyl, or —(CH₂)ₘ—R61. The moiety is an “oxime” when R is H; and it is an “oxime ether” when R is alkyl, cycloalkyl, alkenyl, alkynyl, aryl, aralkyl, or —(CH₂)ₘ—R61.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 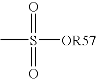
in which R57 is an electron pair, hydrogen, alkyl, cycloalkyl, or aryl.

The term “sulfate” is art recognized and includes a moiety that may be represented by the general formula: 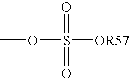
in which R57 is as defined above.

The term “sulfonamido” is art recognized and includes a moiety that may be represented by the general formula: 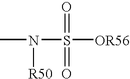
in which R50 and R56 are as defined above.

The term “sulfamoyl” is art-recognized and refers to a moiety that may be represented by the general formula: 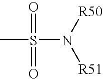
in which R50 and R51 are as defined above.

The term “sulfonyl” is art-recognized and refers to a moiety that may be represented by the general formula: 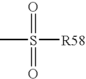
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 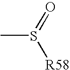
in which R58 is defined above.

The term “phosphoryl” is art-recognized and may in general be represented by the formula: 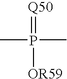
wherein Q50 represents S or O, and R59 represents hydrogen, a lower alkyl or an aryl. When used to substitute, e.g., an alkyl, the phosphoryl group of the phosphorylalkyl may be represented by the general formulas: 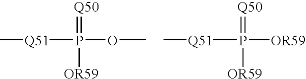
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 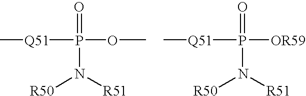
wherein Q51, R50, R51 and R59 are as defined above.

The term “phosphonamidite” is art-recognized and may be represented in the general formulas: 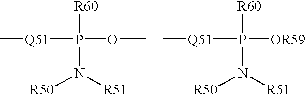
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by o rganic c hemists of ordinary skill in the art appears in the first issue of each volume of the Journal of Organic Chemistry; this list is typically presented in a table entitled Standard List of Abbreviations.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991). Protected forms of the inventive compounds are included within the scope of this invention.

For purposes of this invention, the chemical elements are identified in accordance with the Periodic Table of the Elements, CAS version, Handbook of Chemistry and Physics, 67th Ed., 1986-87, inside cover.

### Synthesis and Characterization of poly(allylamine)-g-polyr(ethylene glycol)

A graft copolymer with an electrostatic polymer backbone and PEG side chains was synthesized through the modification of commercially available poly(allylamine); this polymer was synthesized through the reaction between N-hydroxysuccinimide (NHS) ester and primary amine groups along the poly(allylamine) backbone resulting in the formation of amide bonds with the NHS groups leaving, as shown in Scheme 1.

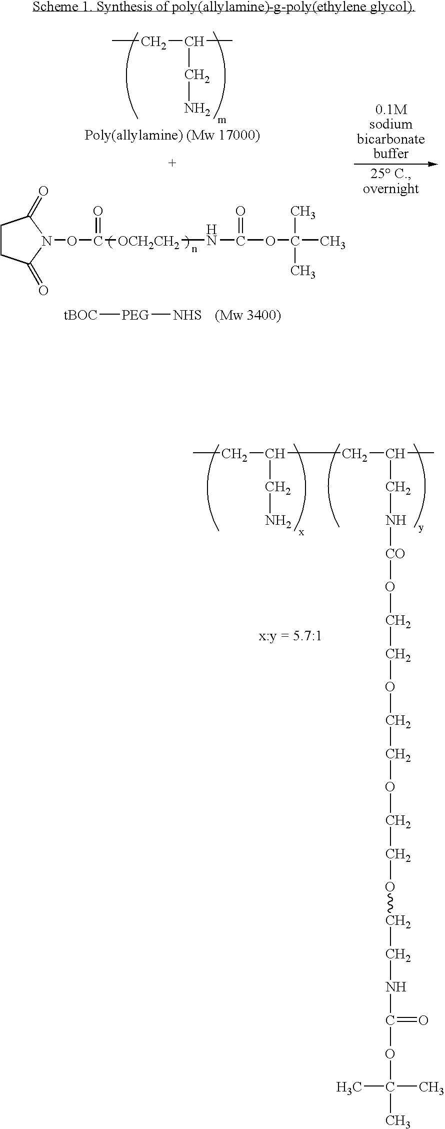

The remaining nonfunctionalized amine groups on the backbone serve as the basis for adhesion of the entire graft copolymer to a negatively charged substrate. The degree of ionization of the graft copolymer backbone can be tuned by adjusting the pH of the aqueous polymer solution, which can be used to vary the thickness and density of functional graft groups of dip-coated or POPS transferred layer of the graft copolymer. The hetero-functional primary amine group on PEG side chain was available originally in the tBoc protected form in order to prevent the auto-condensation reaction with NHS ester group at the other end. After the grafting, tBoc protection can be removed in neat trifluoroacetic acid to restore primary amine groups at side chain ends for further uses as depicted in Scheme 2.

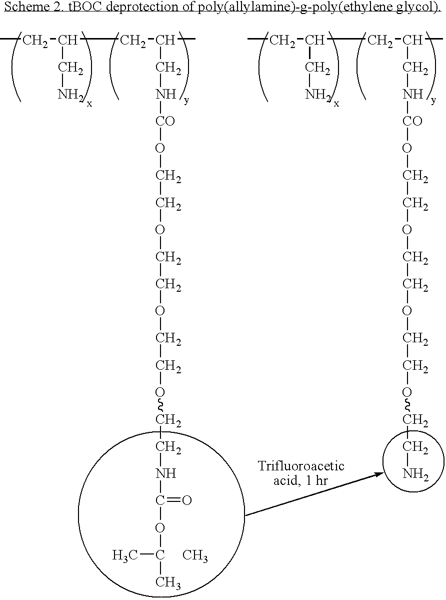

The length of the PEG side chains can be varied; in this work, a molecular weight of 3400 was chosen, which ensures a strong protein resistance due to entropic and hydration effects.

GPC analysis of the graft copolymer revealed a weight averaged molecular weight of 148,000 and a polydispersity index (Mw/Mn) of 2.8 for the tBoc protected graft copolymer. After deprotection, the molecular weight was lowered to 124,000 and the polydispersity index increased to 2.9. Removal of the tBoc group was verified with NMR, based on the disappearance of tBoc hydrogen (—C(CH₃)₃) peak at 1.32 ppm. No evidence of chain scission was detected in GPC or NMR data after cleavage of the tBoc protection groups in the highly acidic solvent, trifluoroacetic acid.

Based on GPC data, the calculated average fraction of fuictionalized repeat units along poly(allylamine) backbone, the grafting density, was about 13%, and the composition ratio, weight of side chains to weight of backbone, was about 7.7. These calculations were based on the assumption that molecular weight of poly(allylamine) is 17,000 as given by the manufacturer. The RI detector of GPC did not give any signal for poly(allylamine) itself in 0.8 M sodium nitrate buffer possibly because the polymer was stuck inside the column and did not elute. Although Mw 17,000 of poly(allylamine) is on PEG standard basis, it was taken as the absolute molecular weight of poly(allylamine) to calculate the number of repeat units of the polymer, which is about 300.

Determination of the grafting density of the copolymer using proton NMR spectra was attempted. However, due to the congestion and broad shape of peaks, it was difficult to draw the grafting density out of NMR data. Instead, a second estimate of the grafting density using zeta potential values was obtained. Two assumptions were required: zeta potential is determined only by the amount of charged amine groups in the molecule; and amine groups on the backbone and at the end of the side chains contribute to the zeta potential to the same extent.

Zeta potential of the tBoc protected graft copolymer molecule was +4.24±1.99 (mV) and it increased to +5.16±1.03 (mV) after the removal of tBoc protecting group. A grafting density of 18% was calculated from the 18% decrease in zeta potential of tBoc protected graft copolymer compared to the deprotected graft copolymer. Zeta potential is known to be affected by many system parameters, such as viscosity of media, size of molecule, and the hydrodynamic interaction between media and molecule. Thus, the calculation is subject to uncertainty particularly in view of the first assumption. Thus, the grafting density calculated from zeta potential is taken as an order of magnitude crosscheck of the GPC based value of 13%.

Protein Adsorption

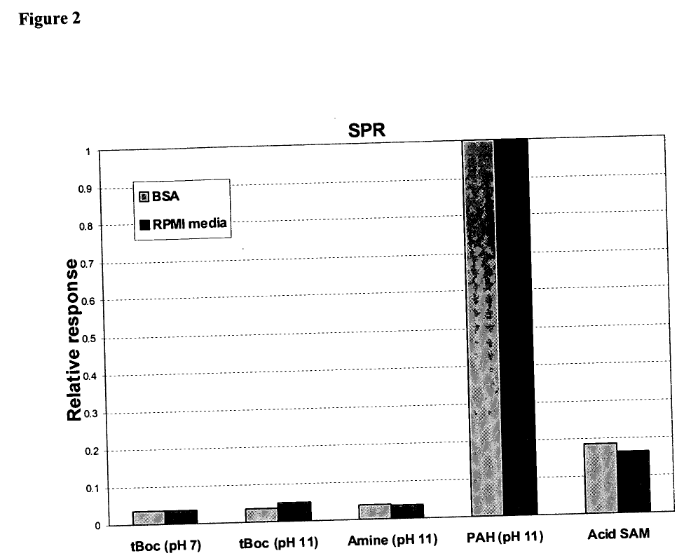

To determine the degree of protein absorption on the graft copolymer surface, surface plasmon resonance was used. Results of protein adsorption experiments are all summarized in FIG. 2. Each ΔRU value was normalized by ΔRU value of PAH surface and this relative value was plotted in FIG. 2. RPMI cell culture media was employed in addition to BSA to study the adsorption characteristics of other proteins since RPMI cell culture media contains the whole bovine serum, not only albumin at 5 wt % concentration. Also it was taken relevant to study protein adsorption in RPMI media that was used in B cell culture because the graft copolymer would be used under this media in B cell array fabrication application. RU signals reached steady state values within 6 minutes of protein solution running periods and within 12 minutes of PBS buffer running periods with the exception of carboxylic-acid-terminated SAM surface, which experienced slight linear increase to the end of protein adsorption period but steady state values within 12 minutes of PBS buffer running period. Due to the large uptake of proteins, the PAH surface exhibited slow increase in early stage of protein adsorption, but reached steady state values in about 3 minutes.

The graft-copolymer-coated sensor chips exhibited a substantial decrease of protein adsorption compared to a PAH coated surface or a carboxylic acid terminated SAM surface in both cases of BSA and RPMI cell culture media, even though the magnitude of resonance unit change (ΔRU) in the RPMI cell culture media adsorption was larger than in BSA adsorption due to a higher concentration of proteins in RPMI media. ΔRU in BSA adsorption for the graft copolymer coated surface ranged from 119 to 132 while in RPMI cell culture media it increased from 202 to 280. 1 ΔRU is roughly equivalent to adsorption of 1 pg/mm² for most proteins.

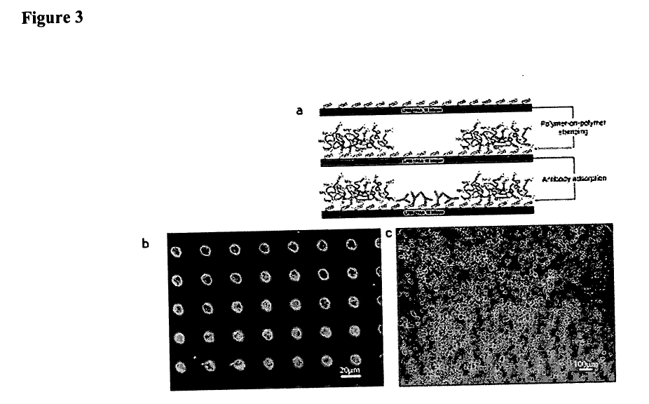

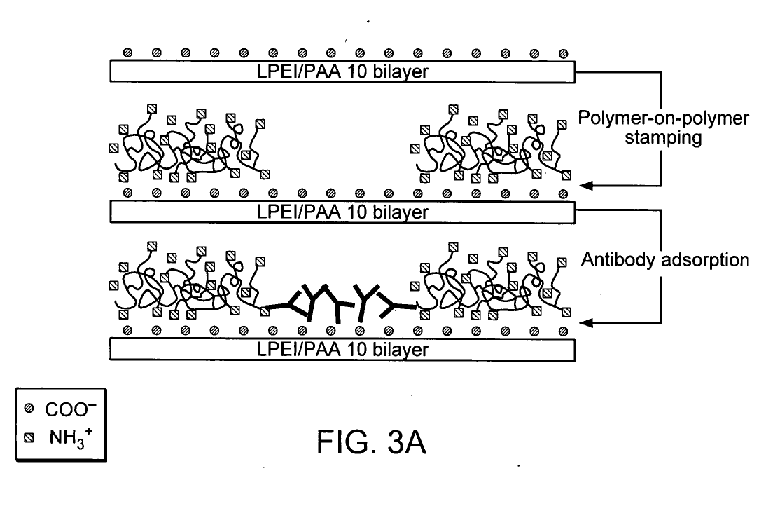

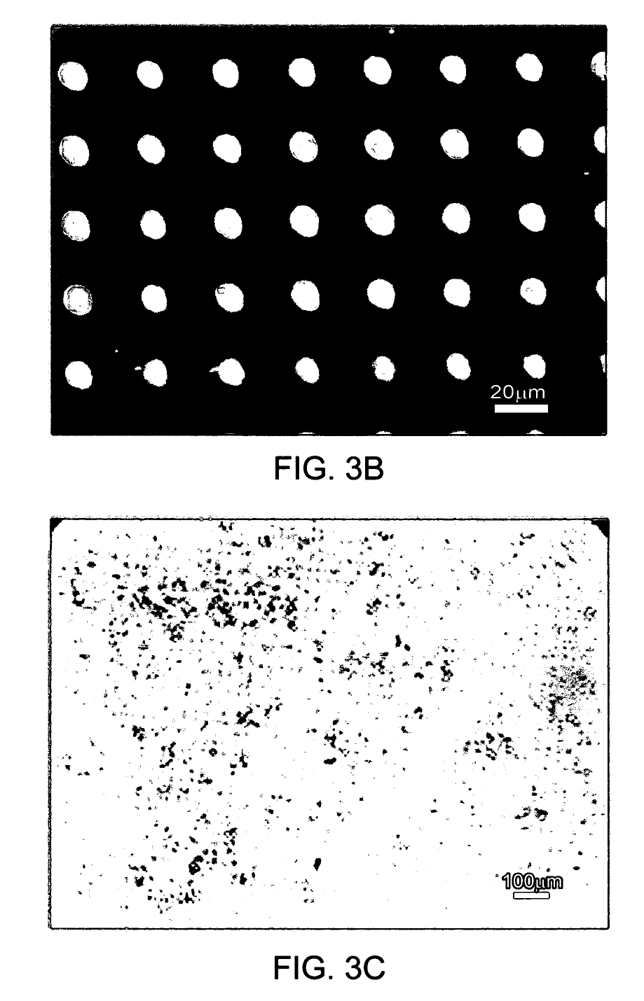

Even under different experimental conditions for these two media, plotting relative responses reveal the same trends between two cases in FIG. 2. Since other protein components in serum, such as immunoglobulins, are different from BSA in size and electrostatic characteristics, contributions from other proteins in RPMI media would have resulted in a different adsorption behavior compared to BSA, at least on PAH and carboxylic acid SAM surfaces. The same trends between the two cases, however, suggest albumin dominates the competitive adsorption in the whole serum and effects of other proteins are negligible. Supportive results of competitive adsorption study of a mixture of albumin, immunoglobulin, and fibrinogen are found in literature. In the study, the smaller protein, albumin, that was at higher concentration in the bulk, dominated the early stage protein adsorption on polystyrene surface and was replaced by other larger proteins slowly. In our experiment, the adsorption was allowed only for 6 minutes, which is not long enough to observe the substitution of adsorbing species. In addition to the dominance of albumin in the competitive adsorption kinetics, early saturation of adsorption sites on surface may be responsible for the same trends between the BSA and the whole serum proteins. It suggests the saturation of adsorption sites on the surface that 50 times higher concentration of proteins in RPMI media than 0.1 wt % BSA solution used in the experiments resulted in only about twice increase of protein adsorption. Consequently, SPR experiment successfully demonstrated the adsorption resistance of the graft copolymer to BSA in single and competitive modes. The effectiveness to other kinds of proteins was confirmed qualitatively, where the graft copolymer w as stamped and dip-coated to c reate the adsorption resist region to antibody and streptavidin, respectively. See FIG. 3.

The grafting of poly(ethylene glycol) onto poly(allylamine) did not compromise the protein adsorption resistance of PEG nor the electrostatic binding capability of poly(allylamine). As table protein adsorption resistant coating was created simply by the dipping of negatively charged surface (carboxylic acid terminated SAM) into the aqueous solution of the graft copolymer. Adsorption of proteins on a poly(allylamine) coated surface was reduced to less than 5% of the original value by the introduction of PEG side chains. Deprotection of tBoc groups had little influence on the adsorption resistance of the graft copolymer. The electrostatic interactions between proteins and amine end groups of PEG are evidently not large enough to counteract effectively the inherent hydrated-state protein adsorption resistance of PEG; furthermore, some of the amine groups generated by elimination of tBoc groups may bind to the surface, yielding PEG side chain loops as a brush layer and thereby effectively decreasing the number of amine groups available to interact with proteins.

Protein adsorption resistance of the graft copolymer coated surface was also investigated through the attachment study of protein mediated binding cells. Inhibition of fibroblast (NR6 WT) attachment was verified on the graft copolymer surface as generally reported with PEG coated surface.

Patterning

The patterned arrays of antibody were generated via the polymer-on-polymer stamping (POPS) of graft copolymer, poly(allylamine)-g-poly(ethylene glycol) (FIG. 1a). Jiang, X. P.; Zheng, H. P.; Gourdin, S.; Hammond, P. T. Langmuir 2002, 18, 2607-2615. Compared to the microcontact printing of self-assembled monolayers, for which small oligomer molecules are transferred onto metal or inorganic surfaces via covalent bond formation, polymeric materials are stamped onto the polymer surface in POPS through electrostatic, van der Waals, or hydrogen-bonding interactions, as well as covalent bonding. Xia, Y. N.; Whitesides, G. M. Annu. Rev. Mater. Sci. 1998, 28, 153-184; Jiang, X. P.; Zheng, H. P.; Gourdin, S.; Hammond, P. T. Langmuir 2002, 18, 2607-2615. A diverse range of materials with numerous types of functionality and structure can be transferred to form a stable patterned layer on a charged surface, including the surface of a polyelectrolyte multilayer with tailored optical, electrical, or surface properties. The great versatility of the graft copolymer used as an ink material in POPS comes from multiple features of the structure. The major components of the graft copolymer are the poly(ethylene glycol) (PEG) comb branches, which comprise 90% wt of the polymer. PEG is well known to possess protein adsorption resistance at aqueous interfaces, and to act as a barrier to nonspecific cell attachment. Michel, R.; Lussi, J. W.; Csucs, G.; Reviakine, I.; Danuser, G.; Ketterer, B.; Hubbell, J. A.; Textor, M.; Spencer, N. D. Langmuir 2002, 18, 3281-3287; Csucs, G.; Michel, R.; Lussi, J. W.; Textor, M.; Danuser, G. Biomaterials 2003, 24, 1713-1720; Gombotz, W. R.; Guanghui, W.; Horbett, T. A.; Hoffinan, A. S. J. Biomed. Mater. Res. 1991, 25, 1547-1562. On the other hand, the polycation backbone of the polymer, which is based on poly(allylamine hydrochloride) (PAH), facilitates transfer of the polymer onto a negatively-charged surface, such as silicon oxide, or a negatively charged polyelectrolyte multilayer, to form a very stable and uniform polymer layer. Because only a small portion of the amine groups of poly(allylamine) are protonated at the pH used for stamping, there are many free amine groups left after it binds electrostatically to a negatively-charged surface. Berg, M. C.; Choi, J.; Hammond, P. T.; Rubner, M. F. Langmuir 2003, 19, 2231-2237. These free amine groups are available for surface reaction and their density can be adjusted simply by changing pH of the PAH-g-PEG copolymer solution. Consequently, these features enable the creation of a surface pattern derivatized with various functionalities with the simultaneous suppression of non-specific interactions of other molecules with the surface. In this study, PEG comb branches with tBoc-termini and amino-termini were used, but the variation in PEG termini did not significantly affect the properties of the PAH-g-PEG copolymer. All data shown were obtained using the graft copolymer with amino-termini.

To provide a specific anchor for B cells, an anti-CD44 antibody was selected and immobilized on the substrate. The antibody array was prepared by the direct adsorption of the antibody onto the patterned regions of a surface not coated by the graft copolymer. To accomplish this system, PAH-g-PEG graft copolymer was stamped onto the negatively charged surface of a polyelectrolyte multilayer (10 bilayers of linear poly(ethylenimine) (LPEI)/poly(acrylic acid) (PAA)). The resulting surface comprised alternating regions of PEG graft copolymer and 10 μm circles of negatively charged PAA. Antibody was allowed to adsorb onto the pattern; due to the protein resistance of the graft copolymer, antibody adsorption could occur only on the circular PAA exposed regions (FIG. 3a). As described above, the amino-termini of the PEG side chains do not support protein binding to regions coated by the graft copolymer. Stability, uniformity, and bio-inertness of the graft copolymer pattern can be inferred from the fluorescence image (FIG. 3b) of the antibody array obtained as depicted in FIG. 3a. B cell array fabrication was completed with simple washing following the seeding of B cells onto the antibody template.

Although PEG side chains dominate the composition of the graft copolymer by the above mentioned factor of 7.7, the graft copolymer was successfully transferred by POPS to the negatively-charged multilayer surface. Methoxy terminated PEG side chains of larger molecular weight (Mw 5,000) have been grafted on poly(allylamine) at much higher grafting density of about 50%. Even at this high composition of PEG (factor of 44.1), the graft copolymer was successfully patterned via POPS. In a control experiment, poly(ethylene glycol) of molecular weight 10,000 was directly stamped under the same condition and no evidence of pattern transfer was found in optical microscopy and AFM scanning following vigorous rinsing procedures.

These experiments suggest the important role of the polycationic backbone in providing the capability to achieve strong binding of the polymer to negatively charged surfaces.

Surface Derivatization

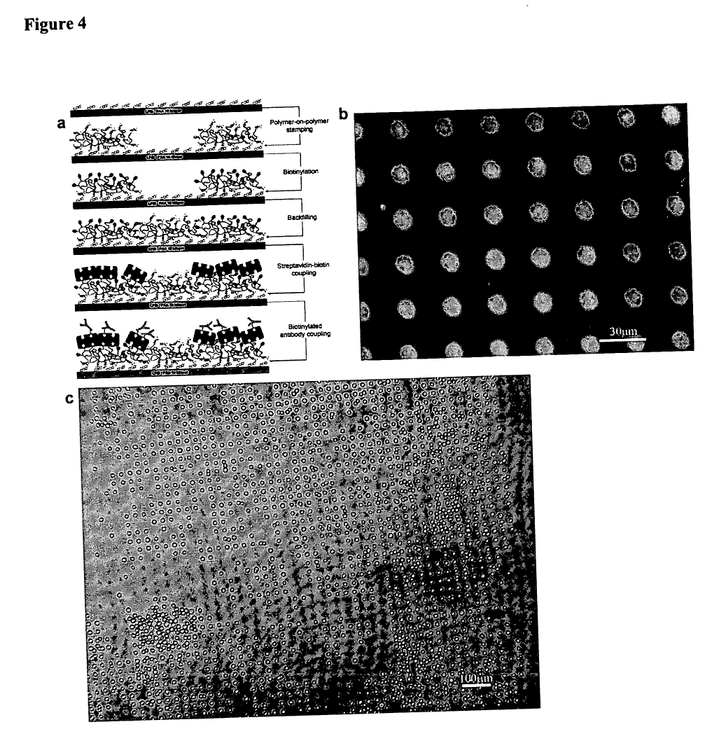

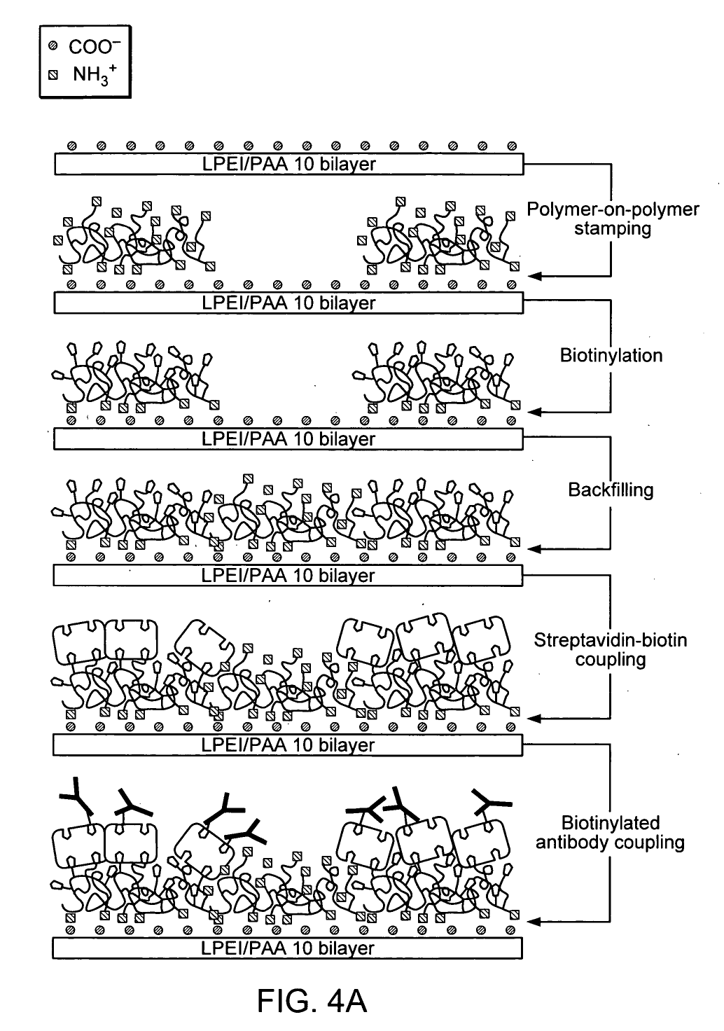

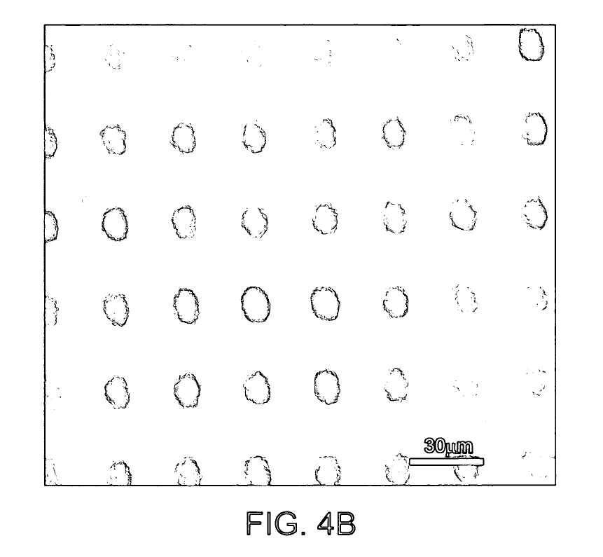

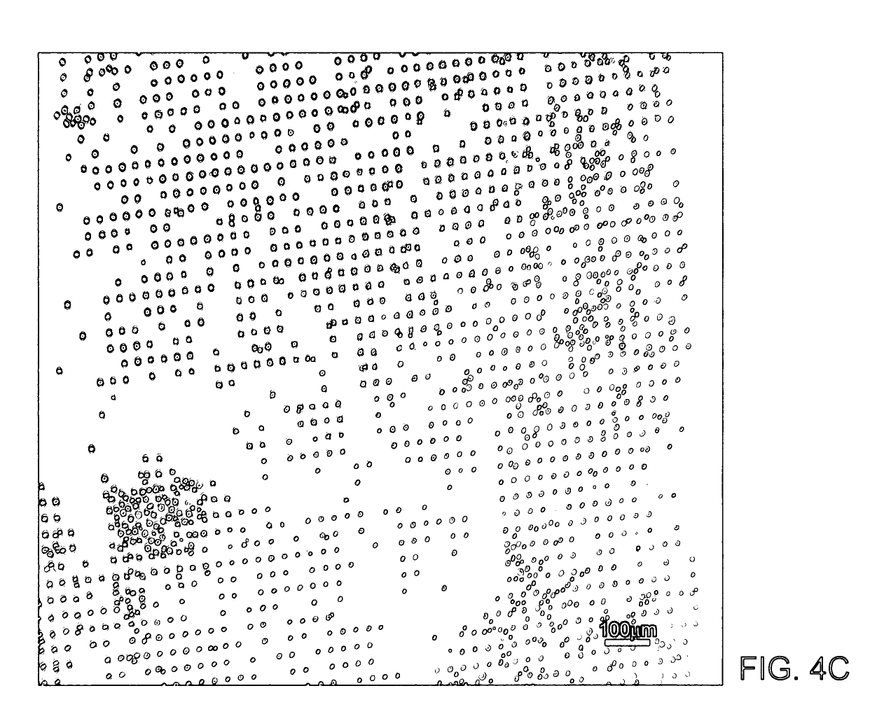

Biotin functional derivative of the graft copolymer was made by surface reaction as described in experimental section. Since amidation of NHS activated carboxylic acid is more rapid with primary amine than with secondary amine, biotinylation of underlying LPEI was negligible. Biotinylation was possible for the graft copolymer with tBoc protection groups, as well as the deprotected graft copolymer. Biotinylation was indirectly confirmed by specific interactions of biotin with streptavidin. Fluorescence-tagged streptavidin was used to detect the coupling of biotin with streptavidin, and patterns of the graft copolymer were used in biotinylation in order to achieve fluorescence contrast under microscope after streptavidin binding. A backfilling step followed biotinylation in order to prevent non-specific adsorption of streptavidin on un-biotinylated areas. These procedures are schematically summarized in FIG. 4a and the resulting streptavidin pattern is shown in FIG. 4b. RGD ligand immobilization on the patterned surface was also performed by surface reaction as described in the experimental section. The reaction between the sulfo-SMCC linker and the graft copolymer was based on the same NHS chemistry as sulf-NHS-LC-biotin and the same reactivity issue with LPEI could be addressed in the same way. The reaction between free sulfhydryl groups in cysteine (RGDEC) and maleimide groups of the linker continued overnight in dark condition to prevent photobleaching of dansyl chloride dye. Unreacted oligopeptides were removed by ultrasonification in deionized water. The reaction was also indirectly confirmed by fluorescence from dansyl chloride and patterns of the graft copolymer were used in RGD immobilization in order to achieve fluorescence contrast under microscope. The contrast in fluorescence was detected between the maleimide linker conjugated region and the region backfilled after the maleimide conjugation. Similar results were obtained with the graft copolymer with tBoc protection groups, as well as the deprotected graft copolymer.

In both cases, amine groups on the graft copolymer backbone, as well as those at the ends of the PEG side chains, are available for amidation, the reaction accompanies our biotinylation and RGD immobilization schemes, as indicated from the tBoc protected graft copolymer derivatization. In the biotinylation case, although PEG side chains are much longer than the arms of biotin, streptavidin was successfully bound to the biotins on the graft copolymer despite the PEG brush layer while they could not adsorb non-specifically on the PEG brush layer without biotin moieties around. Since the poly(allylamine) is a weak polyelectrolyte, at pH 11 some of un-ionized fraction of amine groups might be exposed out of PEG brush layer by loopy chain conformation of poly(allylamine) on the negatively charged substrate. The fraction of exposed backbone out of PEG brush layer might not be large enough to induce non-specific interaction of large protein molecules while remaining accessible to small linker molecules such as biotin. Once biotinylated, the great avidity o f biotin and streptavidin binding might allow streptavidin to bind to biotin and sit on the graft copolymer layer. This might not be an issue in RGD ligand immobilization due to the small size of oligopetide. The conformational variation of weak polyelectrolytes that is caused by the change of the ionization fraction at different pH on adsorption can be exploited to gain a simple way to control the extent of surface functionalization. In poly(allylamine) case, for example, less amine groups are protonated at high pH and more amine groups remain available for surface reaction after the adsorption. The capability of adjusting the number of available amine groups provides an easy way to optimize the extent of functionalization. In solution state reaction, however, a change of solution pH may cause the reactivity decrease of the other reagent. For instance, NHS-activated carboxylic acid is subject to more hydrolysis at higher pH. That is, less reactive NHS chemistry would counterbalance larger amount of deprotonated amine groups on poly(allylamine) at high pH; the extent of reaction would not be simply controlled by the adjustment of pH. The same discussion can be extended to the graft copolymer with deprotected amine groups. Some of protonated amine groups at PEG side chain ends may participate in surface binding and loop formation as discussed in the protein adsorption section.

Compared to the first antibody array template, introduction of the biotin linker on the antibody can give rise to greater orientational freedom of the antibody, resulting in more effective array templates (FIG. 4b). Cellular arrays on this template were greatly improved over the first case (FIG. 4c). Even after intensive washing to remove clustered cells, many of the arrayed cells remained. This type of array surface, utilizing a streptavidin-biotin interaction to bind antibodies to the substrate, can be easily generalized to a large number of cell types simply by altering the choice of biotinylated antibody used.

B Cell Array Fabrication

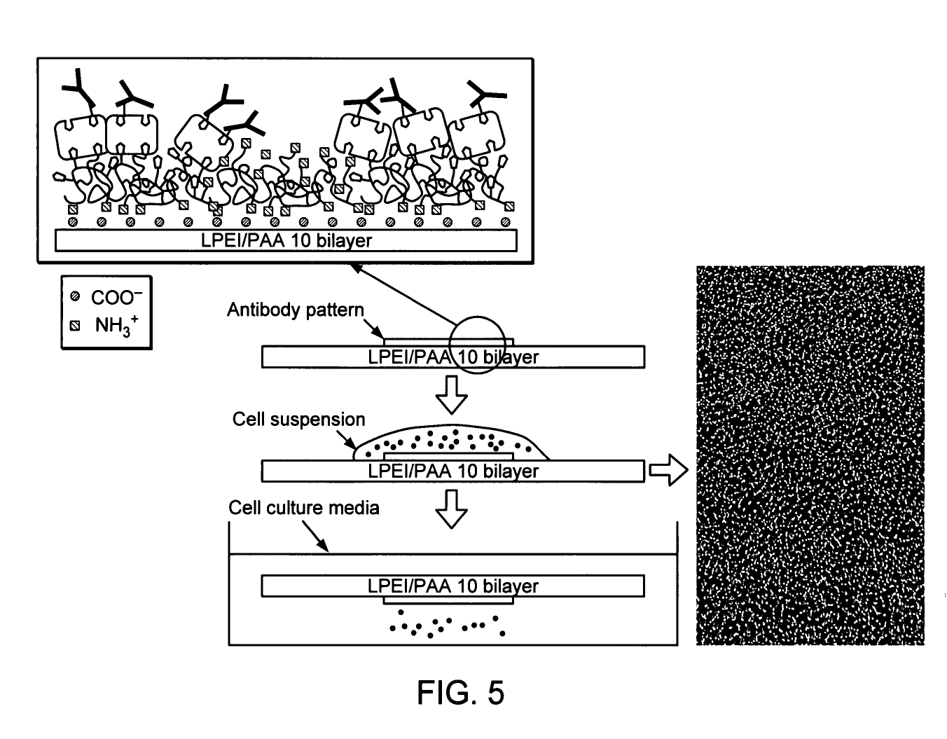

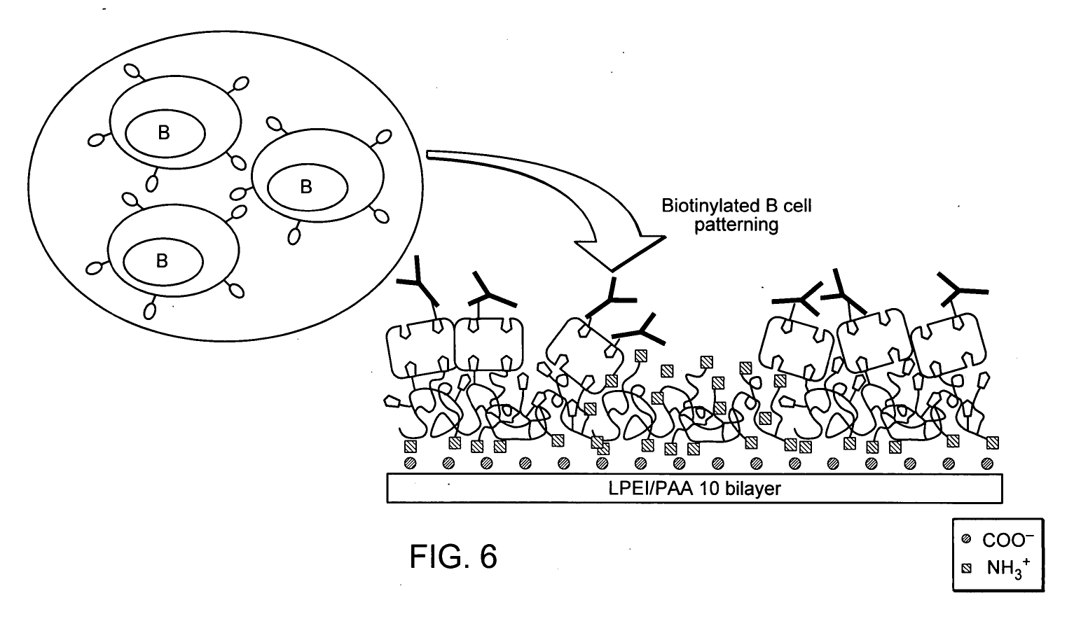

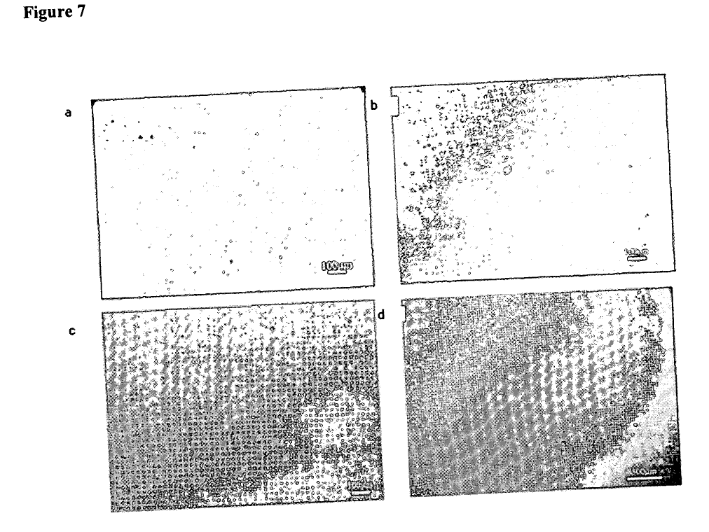

To provide a specific anchor for B cells, an anti-CD44 antibody was selected and immobilized on the substrate; unlike many other cell types studied for the similar purpose, B cells do not express adhesion receptors and thus do not adhere appreciably to the surface. As B cell array fabrication template, 10 μm dot arrays of antibody were prepared by two different methods. The first template was produced by simple adsorption of antibody on the graft copolymer patterned surface (FIG. 3c). The second was made starting from biotinylated graft copolymer pattern via streptavidin coupling as depicted in FIG. 3a. The coupling of biotinylated antibody with streptavidin array pattern (FIG. 3b) generated an antibody array as delineated schematically in FIG. 3a. B cell arrays were fabricated on these antibody array templates as presented schematically in FIG. 5. Biotinylated B cells were also used with the second antibody array template as depicted schematically in FIG. 6. Of these B cell arrays, biotinylated B cells on the second template resulted in the best quality of clean large area array. The results are presented in FIG. 7.

In the case of the second template, introduction of the biotin linker on the antibody results in greater orientational freedom of the antibody, resulting in more effective array templates. Cellular arrays on this template were greatly improved over the first case (FIG. 7b). Even after more intensive washing to remove clustered cells, many of the arrayed cells remained. The importance of binding strength on pattern fidelity became clearer with the use of biotinylated B cells. The biotin molecules on the cell membrane can participate in surface binding via conjugation with unoccupied streptavidin binding sites and this enhanced binding led to a near perfect, clean array of B cells over a large area (FIG. 7c, d) even at twice lower cell seeding density. Biotinylated B cell arrays of comparable quality were also achieved with the use of streptavidin array templates without antibody.

Applications

The arrays of non-adhesive cells disclosed herein and the methods of preparing them have long range applications in the fields of biosensors and cell research. In addition, the B cell arrays of the present invention should aide in determining the role these cells play in the human body's immunological response system. The following applications represent just a few examples of the possibilities envisioned by the inventors.
Imaging-based high-throughput cellular analysis system. D. Taylor et al. Curr. Opin. Biotechnol., 2001, 12, 75-81; R. Kapur et al. Biomed. Microdevices, 1999, 2, 99-109.
Platform for rare event detection. S. Kraeft, et al. Clin. Cancer Res. 2000, 6, 434-442.
Ultra sensitive cell-based biosensors. Rider, T. H. et al. Science 2003, 301, 213-215.
Fundamental studies of cell biology. G. Whitesides, et al. Annu. Rev. Biomed. Eng. 2001, 3, 335-373.

### EXEMPLIFICATION

The invention now being generally described, it will be more readily understood by reference to the following examples, which are included merely for purposes of illustration of certain aspects and embodiments of the present invention, and are not intended to limit the invention.

Materials—Poly(allylamine) (Mw 17,000, 20% in aqueous solution) from Sigma-Aldrich Co., St. Louis, Mo., tBoc-NH-poly(ethylene glycol)-N-Hydroxysuccinimide (Mw 3,400) from Nektar Therapeutics, H untsville, A L, Linear p oly(ethyleneimine) (Mw 2 5,000) and poly(acrylic acid) (Mw 90,000, 25% in aqueous solution) from Polysciences Inc., Warrington, Pa., sulfosuccinimidyl-4-(N-maleimidomethyl)cyclohexane-1-carboxylate (sulfo-SMCC) and sulfosuccinimidyl-6-(biotinamido) hexanoate (sulfo-NHS-LC-biotin) from Pierce Biotechnology Inc., Rockford, Ill., RGDEC peptide sequence with dansyl chloride from Biopolymers Laboratory at MIT, CD44:FITC from BD Biosciences, San Diego, Calif., and streptavidin from Molecular Probes, Eugene, Oreg. were purchased and used as received.

GPC and NMR—All GPC measurements were run in a 0.8 M sodium nitrate aqueous buffer. The equipment was calibrated with PEG standard. NMR analysis of the graft copolymer was performed in D₂O solvent at 400 MHz.

Zeta Potential Measurement—The graft copolymer was dissolved to 1 wt % in deionized water without any pH adjustment. ZetaPALS instrument (Brookhaven Instrument Co., Holtzville, N.Y.) was used to measure the zeta potentials of the graft copolymer solutions.

Surface Plasmon Resonance Spectroscopy—Biacore 1000 SPR instrument (Biacore, N.J.) was used for the study of protein adsorption on graft copolymer coated surfaces. The sensor chip substrate was made by the evaporation of titanium as an adhesion layer (1 nm) followed by a gold layer of 40 nm on cover glass slips of 0.2 mm. Gold coated cover glass was cut into a proper size for the sensor chip holder. Carboxylic acid terminated self-assembled monolayer (SAM) was produced on the chip surface by immersing the gold coated substrate into 2 mM solution of mercaptohexadecanoic acid in ethanol for 3 hours. After rinsing with ethanol and blow-drying with air, the chip substrate was mounted on the holder with the use of epoxy glue. Carboxylic acid terminated SAM surface of the chip was coated with the graft copolymer or poly(allylamine hydrochloride) (PAH) (Mw 90,000, Sigma-Aldrich Co., St. Louis, Mo.) by dipping the chip into 10 mM aqueous solution of each polymer for 15 minutes and allowing adsorption to occur based on electrostatic interactions. Polymer solution pH was all adjusted to 11; for the graft copolymer with the tBoc protection groups, additional pH 7 solution was prepared to investigate the effect of coating solution pH on protein adsorption resistance.

1 mg/ml bovine serum albumin (BSA) solution in PBS and RPMI cell culture media were used in the protein adsorption experiments. Before the injection of protein solutions into chip microfluidic channels, PBS buffer was applied for 2 minutes to equilibrate the coated polymers to an aqueous environment. Protein solution was injected for 6 minutes, followed by PBS buffer injection for 12 minutes. Difference in resonance units (RU) at the ends of the first injection of PBS buffer for equilibration and the second injection of PBS buffer following the protein injection was taken as resonance unit change (ΔRU) due to protein adsorption. Flow rate was set always at 5 μI/min except for the priming and purging steps.

### EXAMPLE 1

Synthesis of poly(allylamine)-g-poly(ethylene glycol)—0.25 g (equivalent to 4.4 mM of allylamine repeat units) of poly(allylamine) was dissolved in 0.1 M aqueous sodium bicarbonate buffer. 5.0 g (equivalent to 1.5 mM of polymers) of tBoc-NH-PEG-NHS was added to the poly(allylamine) solution. Reaction proceeded overnight under stirring at room temperature. Unreacted species were filtered out using a stirred ultrafiltration cell apparatus (Millipore, Bedford, Mass.) with a molecular weight cut-off filter (polyethersulfone, Mw 10,000, Millipore, Bedford, Mass.). The graft copolymer was retrieved via vacuum distillation.

Removal of the tBoc protecting group was done in neat trifluoroacetic acid (TFA). After 3 hours of stirring, the mixture was diluted with water, neutralized with NaOH, and filtered through the molecular weight cut-off filter.

### EXAMPLE 2

Synthesis of polyelectrolyte multilayer (PEM)—Polyelectrolyte multilayers assembled from the weak polyelectrolytes, linear poly(ethyleneimine) (LPEI) and poly(acrylic acid) (PAA) were used in this study. 10 mM aqueous solution of each polyelectrolyte was prepared. PH of the LPEI solution was adjusted to 7.5 and pH of the PAA solution to 3.5. A glass cover slip was cleaned by ultrasonification in detergent solution for 3 minutes, rinsed vigorously with deionized water, and treated with ultrasonification in deionized water for 3 minutes. Cleaned cover glass slide was immersed in the prepared LPEI solution for 15 minutes and then rinsed three times in deionized water with gentle agitation for 2, 1, and 1 minute(s), respectively. After these 3 steps of rinsing, the positively charged substrate that resulted from the adsorption of polycation, LPEI, was submerged in the prepared polyanion, PAA, solution for 15 minutes. 3 rinsing steps followed in the same manner. Alternating adsorptions of LPEI and PAA built up one bilayer of polycation/polyanion, yielding a negatively charged surface for next polycation layer adsorption. This procedure was continued until 10 bilayers were deposited on the substrate with the top-most layer being PAA.

### EXAMPLE 3

Polymer-on-polymer stamping (POPS)—A 10 mM aqueous solution of the graft copolymer at pH 11 was prepared as ink for POPS. A PDMS stamp was immersed in ink solution for an hour to allow the ink polymer to adsorb on PDMS surface. Subsequently, the stamp was gently rinsed with deionized water and blow-dried with air. The dried stamp was then placed on the negatively charged multilayer substrate. After 2 minutes of contact with the inked stamp, the substrate was vigorously rinsed with deionized water to remove excess material loosely bound on the substrate. The stability of stamped layer was tested by ultrasonification for 2 minutes in deionized water.

### EXAMPLE 4

Antibody adsorption—The substrate patterned with the graft copolymer by polymer-on-polymer stamping was immersed in 1 μg/ml solution of fluorescence labeled antibody of CD44:FITC for 10 minutes and then rinsed in PBS buffer for 2 minutes under ultrasonication. After blow-drying the substrate with air, antibody adsorption on the patterned surface was examined via fluorescence microscopy.

### EXAMPLE 5

RGD functionalization—A 1 mM solution of sulfo-SMCC was prepared in PBS buffer. The POPS-patterned substrate was submerged in the prepared sulfo-SMCC solution. After an hour of reaction between sulfo-SMCC and amine groups of the patterned graft copolymer, the substrate was washed with deionized water and blow-dried with air. The substrate was immersed in the 10 mM graft copolymer aqueous solution for 15 minutes to backfill the unstamped area. The substrate was rinsed with deionized water and blow-dried with air.

A 0.5 mM solution of dansyl chloride-RGDEC peptide sequence was prepared in PBS buffer. The substrate, after maleimide linker conjugation and backfilling with the graft copolymer, was dipped in the oligopeptide solution. The reaction proceeded overnight at room temperature and was followed by rinsing for 2 minutes in deionized water under ultrasonification.

### EXAMPLE 6

Surface biotinylation and streptavidin coupling—A 1 mM solution of sulfo-NHS-LC-biotin was prepared in PBS buffer. The POPS-patterned substrate was submerged in the prepared sulfo-NHS-LC-biotin solution. After an hour of reaction between sulfo-NHS-LC-biotin and amine groups of the patterned graft copolymer, the substrate was washed with deionized water and blow-dried with air. The substrate was immersed in the 10 mM graft copolymer aqueous solution for 15 minutes to backfill the unstamped area. The substrate was rinsed with deionized water and blow-dried with air.

Streptavidin solution (1 μg/ml) was prepared in PBS buffer. The substrate, after biotinylation and backfilling with the graft copolymer, was dipped in the streptavidin solution for 10 minutes, rinsed with PBS buffer, and stored in PBS buffer to minimize denaturation of streptavidin until further use.

### EXAMPLE 7

Antibody and B cell biotinylation—5 mg of sulfo-NHS-LC-biotin was added to 0.5 mg/ml antibody solution in PBS buffer. After 2 hours of reaction at 4° C., the mixture was dialyzed to remove unreacted biotins.

25×10⁶ B cells were rinsed three times with PBS before biotinylation to remove extra proteins originating from cell culture media and then suspended in 1 ml PBS buffer. 0.5 mg of sulfo-NHS-LC-biotin was added into the prepared B cell suspension. After the 30 minutes of reaction at room temperature, B cells were rinsed three times with PBS and suspended in RPMI cell culture media.

### EXAMPLE 8

B cell array—A few drops of B cell suspension containing enough cells to cover the patterned area were put on the patterned substrate. 20 minutes after all B cells precipitated down on the surface, the substrate was immersed cell side up very gently in the cell culture media and then flipped over within the RPMI cell culture media. It remained upside down in the RPMI cell culture media for about 5 minutes, allowing unbound cells to fall off from the substrate. Sometimes the substrate was gently shaken in the culture medium to get rid of extra cells.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. An array of non-adhesive cells, comprising:
a) a polymeric substrate;
b) an array of a graft copolymer bound to the polymeric substrate;
c) an antibody bound to the polymeric substrate in an area of the polymeric substrate not covered by the graft copolymer; and
d) a non-adhesive cell bound to the antibody.

2. The array of claim 1, wherein the polymeric substrate comprises bilayers of two different polymers.

3. The array of claim 1, wherein the polymeric substrate comprises bilayers of two different polymers, wherein one polymer is linear poly(ethylenimine) and the other one is poly(acrylic acid) (PAA).

4. The array of claim 1, wherein the graft copolymer comprises poly(allylamine).

5. The array of claim 1, wherein the graft copolymer comprises poly(ethylene glycol).

6. The array of claim 1, wherein the graft copolymer is poly(allylamine)-g-poly(ethylene glycol).

7. The array of claim 1, wherein the antibodies are CD44:FITC.

8. The array of claim 1, wherein the non-adhesive cells are lymphocyte or stem cells.

9. The array of claim 1, wherein the non-adhesive cells are B cells.

10. The array of claim 1, wherein the non-adhesive cells are CH27 B cells.

11. An array of non-adhesive cells, comprising:
a) a polymeric substrate;
b) an array of a biotinylated graft copolymer bound to the polymeric substrate, wherein the biotin is bound to a protein having a high affinity for biotin;
c) a graft copolymer free of biotin bound to an area of the polymeric substrate not covered by the array of biotinylated graft copolymer;
d) a biotinylated antibody bound to the protein; and
e) a non-adhesive cell bound to the antibody.

12. The array of claim 11, wherein the polymeric substrate comprises bilayers of two different polymers.

13. The array of claim 11, wherein the polymeric substrate comprises bilayers of two different polymers, wherein one polymer is linear poly(ethylenimine) and the other one is poly(acrylic acid) (PAA).

14. The array of claim 11, wherein the biotinylated graft copolymer is a graft copolymer.

15. The array of claim 11, wherein the biotinylated graft copolymer comprises poly(allylamine).

16. The array of claim 11, wherein the biotinylated graft copolymer comprises poly(ethylene glycol).

17. The array of claim 11, wherein the biotinylated graft copolymer is biotinylated poly(allylamine)-g-poly(ethylene glycol).

18. The array of claim 11, wherein the graft copolymer free of biotin comprises poly(allylamine).

19. The array of claim 11, wherein the graft copolymer free of biotin comprises poly(ethylene glycol).

20. The array of claim 11, wherein the graft copolymer free of biotin is poly(allylamine)-g-poly(ethylene glycol).

21. The array of claim 11, wherein the protein having a high affinity for biotin is streptavidin.

22. The array of claim 11, wherein the antibody is CD44:FITC.

23. The array of claim 11, wherein the non-adhesive cells are lymphocyte or stem cells.

24. The array of claim 11, wherein the non-adhesive cells are B cells.

25. The array of claim 11, wherein the non-adhesive cells are CH27 B cells.

26. A method of preparing a non-adhesive cell array, comprising:
a) depositing an array of a graft copolymer upon a polymeric substrate;
b) binding an antibody to an area of the polymeric substrate from step a) not covered by the graft copolymer; and
c) binding a non-adhesive cell to the antibody from step b).

27. The method of claim 26, wherein depositing the array of graft copolymer comprises POPS.

28. The method of claim 26, wherein binding an antibody to the polymeric substrate comprises immersing the polymeric substrate from step a) into a solution of antibodies followed by rinsing.

29. The method of claim 26, wherein binding a non-adhesive cell to the antibody comprises placing a suspension of the non-adhesive cell over the polymeric substrate from step b);
allowing the non-adhesive cell to precipitate upon the polymeric substrate; and inverting the polymeric substrate allowing any non-bound non-adhesive cells to fall off.

30. The method of claim 26, wherein depositing the array of graft copolymer comprises POPS; binding anitibodies to the polymeric substrate comprises immersing the polymeric substrate from step a) into a solution of antibodies followed by rinsing; and binding non-adhesive cells to the antibodies comprises placing a suspension of the non-adhesive cells over the polymeric substrate from step b); allowing the non-adhesive cells to precipitate down upon the polymeric substrate; and inverting the polymeric substrate allowing the non-bound non-adhesive cells to fall off.

31. The method of claim 26, wherein the polymeric substrate comprises bilayers of two different polymers.

32. The method of claim 26, wherein the polymeric substrate comprises bilayers of two different polymers, wherein one polymer is linear poly(ethylenimine) and the other one is poly(acrylic acid) (PAA).

33. The method of claim 26, wherein the graft copolymer comprises poly(allylamine).

34. The method of claim 26, wherein the graft copolymer comprises poly(ethylene glycol).

35. The method of claim 26, wherein the graft copolymer is poly(allylamine)-g-poly(ethylene glycol).

36. The method of claim 26, wherein the antibody is CD44:FITC.

37. The method of claim 26, wherein the non-adhesive cells are lymphocyte or stem cells.

38. The method of claim 26, wherein the non-adhesive cells are B cells.

39. The method of claim 26, wherein the non-adhesive cells are CH27 B cells.

40. A method of preparing a non-adhesive cell array, comprising:
a) depositing an array of a graft copolymer upon a polymeric substrate;
b) biotinylating the graft copolymer from step a);
c) depositing a graft copolymer upon an area of the polymeric substrate from step b) not covered by the biotinylated graft copolymer;
d) binding a protein having a high affinity for biotin to the biotinylated graft copolymer from step c);
e) binding a biotinylated antibody to the protein from step d); and
f) binding a non-adhesive cell to the antibody from step e).

41. The method of claim 40, wherein depositing the array of graft copolymer comprises POPS.

42. The method of claim 40, wherein biotinylating the graft copolymer comprises placing a solution of sulfo-NHS-LC-biotin over the graft copolymer from step a) followed by rinsing.

43. The method of claim 40, wherein depositing the graft copolymer upon the area of the polymeric substrate from step b) not covered by the biotinylated graft copolymer comprises immersing the polymer substrate into a solution of the graft copolymer followed by rinsing and blow drying.

44. The method of claim 40, wherein binding a protein that has a high affinity for biotin to the biotinylated graft copolymer from step c) comprises immersing the polymer substrate from step c) into a solution of the protein followed by rinsing.

45. The method of claim 40, wherein binding biotinylated antibodies to the protein from step d) comprises immersing the polymeric substrate from step d) into a solution of biotinylated antibodies followed by rinsing.

46. The method of claim 40, wherein binding non-adhesive cells to the antibodies from step e) comprises placing a suspension of the non-adhesive cells over the polymeric substrate from step e); allowing the non-adhesive cells to precipitate down upon the polymeric substrate; and inverting the polymeric substrate allowing the non-bound, non-adhesive cells to fall off.

47. The method of claim 46, wherein the non-adhesive cells are biotinylated.

48. The method of claim 40, wherein:
i. depositing the array of graft copolymer comprises POPS;
ii. biotinylating the graft copolymer comprises placing a solution of sulfo-NHS-LC-biotin over the graft polymer from step a) followed by rinsing;
iii. depositing the graft copolymer upon areas of the polymeric substrate from step b) not covered by the biotinylated graft copolymer comprises immersing the polymer substrate into a solution of the cograft polymer followed by rinsing and blow drying;
iv. binding a protein that has a high affinity for biotin to the biotinylated graft copolymer from step c) comprises immersing the polymer substrate from step c) into a solution of the protein followed by rinsing;
v. binding biotinylated antibodies to the protein from step d) comprises immersing the polymeric substrate from step d) into a solution of biotinylated antibodies followed by rinsing; and
vi. binding non-adhesive cells to the antibodies from step e) comprises placing a suspension of the non-adhesive cells over the polymeric substrate from step e); allowing the non-adhesive cells to precipitate down upon the polymeric substrate; and inverting the polymeric substrate allowing the non-bound, non-adhesive cells to fall off.

49. The method of claim 48, wherein the binding non-adhesive cells to the antibodies from step e) are biotinylated.

50. The method of claim 40, wherein the polymeric substrate comprises bilayers of two different polymers.

51. The method of claim 40, wherein the polymeric substrate comprises bilayers of two different polymers, wherein one polymer is linear poly(ethylenimine) and the other one is poly(acrylic acid) (PAA).

52. The method of claim 40, wherein the graft copolymer is a graft copolymer.

53. The method of claim 40, wherein the graft copolymer comprises poly(allylamine).

54. The method of claim 40, wherein the graft copolymer comprises poly(ethylene glycol).

55. The method of claim 40, wherein the graft copolymer is poly(allylamine)-g-poly(ethylene glycol).

56. The method of claim 40, wherein the protein having a high affinity fro biotin is streptavidin.

57. The method of claim 40, wherein the antibody is CD44:FITC.

58. The method of claim 40, wherein the non-adhesive cells are lymphocyte or stem cells.

59. The method of claim 40, wherein the non-adhesive cells are B cells.

60. The method of claim 40, wherein the non-adhesive cells are CH27 B cells.

61. The method of claim 58, 59 or 60, wherein the non-adhesive cells are biotinylated.

62. A biosensor comprising the array of non-adhesive cells of claim 1 or 11.
